(19) United States
(12) Patent Application Publication
(10) Pub. No.: US 2001/0006777 A1
(43) Pub. Date: Jul. 5, 2001

(54) MONITORING OF LIQUIDS FOR DISEASE-ASSOCIATED MATERIALS

(76) Inventor: HARASH KUMAR NARANG, NEWCASTLE UPON TYNE (GB)

Correspondence Address: KENNETH I KHON, KOHN & ASSOCIATES, 30500 NORTHWESTERN HWY, STE 410, FARMINGTON HILLS, MI, 48334

(21) Appl. No.: 09/408,023

(22) Filed: Sep. 29, 1999

Related U.S. Application Data
(63) Continuation-in-part of application No. PCT/GB98/00374, filed on Feb. 6, 1998.

(30) Foreign Application Priority Data
Apr. 8, 1999 (GB) 9907962.6

(51) Int. Cl. C12Q 1/70; G01N 33/53; G01N 33/567

(52) U.S. Cl. 435/5; 435/7.2

(57) ABSTRACT

A method for monitoring liquids for the presence of disease-associated materials, so as to provide a non-invasive means for the detection of various materials associated with cancer, autoimmune, neuro-degenerative and other disorders. The method provided comprises contacting a sample of the liquid with a solid, non-buoyant particulate material having free ionic valencies so as to concentrate the disease-modified or associated proteins in the sample and then monitoring the resulting disease-modified or associated proteins concentrated on the particulate material.

### CROSS-REFERENCE TO RELATED APPLICATION

The present application is a continuation in part of international application PCT/GB98/00374 filed on Feb. 6, 1998 by the same applicant as the present invention.

### BACKGROUND OF THE INVENTION

The present invention relates to the monitoring of liquids for disease-associated materials and more specifically to the monitoring of liquids for materials associated with autoimmune and other diseases, all using non-invasive means.

At present, the principal methods for monitoring infectious and autoimmune disorders, cancer and the like, such as Alzheimer's disease, multiple sclerosis, spongiform encephalopathies etc. are invasive techniques involving the monitoring of pathological changes in surgically accessible tissue. Similarly, principal methods for monitoring various cancers also involve invasive techniques. Amyloid plaques, for example, are a common neuropathological feature of Alzheimer's disease and would conventionally require invasive surgery in order to be detected, which is generally undesirable. These surgical methods are expensive and time consuming and are often only undertaken when a disease is at an advanced stage.

Spongiform encephalopathies, such as Creutzfeldt-Jakob disease (CJD), Gerstmann-Straussler-Scheinker Syndrome (GSS) and Kuru in humans; scrapie in sheep and goats and bovine spongiform encephalopathy (BSE) in cattle, mink and cats are all transmissible (infective) neuro-degenerative disorders implicating vacuolation of neurons.

At present, the most reliable method of detecting an encephalopathy is histologically, especially by electron microscopy, but this requires brain tissue removed following autopsy of the dead victim. Although neurological examination and electro-encephalographs (EEG) can provide accurate diagnosis in many cases of encephalopathy, there is an urgent need for a definitive test during life, one which can detect the disease during its early stages and which is non-intrusive.

Therefore, an accurate, non-invasive test would provide means to aid in the early detection and diagnosis of various disorders, thereby improving the possibility for the early treatment of the disease, hence potentially increasing the chances of combatting or arresting the disorder.

The protein associated with for example the neuro-degenerative disorder CJD is thought to be a particle termed a “nemavirus”. In contrast to the morphology of a common virus, which has a two layer structure of nucleic acid protected by an outer coat, the nemavirus particle has an unusual three layer structure which comprises:

1. a protein core,

2. single stranded DNA, and

3. a protein coat.

The single stranded DNA is sandwiched between the protein core and the protein coat. Single stranded DNA from scrapie has been partly sequenced and contains a palindromic repeat sequence TACGTA. The scrapie-specific nucleic acid is single stranded DNA and includes the sequence (TACGTA)ₙ where n is at least 6. The basic six unit of this repeat sequence is palindromic, in the sense that a complementary DNA would have the same TACGTA sequence when read in the 5′ to the 3′ direction. The full length sequence of the DNA is not known, but it is suspected that n is very much larger than 6, perhaps of the order of 20 to 30. Although the DNA sequence is scrapie-specific, BSE, scrapie, CJD and other encephalopathies are thought to result from the same protein associated with the neuro-degenerative disorder transferred to another species. It is therefore believed that the TACGTA palindromic sequence appears in all known spongiform encephalopathies and possibly others.

The protein coat has not yet been characterised. The protein core comprises the protease-resistant protein (PrP) which is termed a “prion”. A prion is encoded by a cellular gene of the host and is thought to contain little or no nucleic acid. However, the cellular form of the prion protein is modified into protease-resistant protein (PrP), by an accessory protein , “Nemo Corrupta” coded by single stranded DNA (PESM, 212, 208-224, (1996). This feature distinguishes prions sharply from virions. To date, no prion-specific nucleic acid which is required for transmission of disease has been identified.

Virus-like nemaviruses are tubulofilamentous particles in shape, typically 23-26 nm in diameter. They are consistently detected in the brains of all known spongiform encephalopathies. These particles have a core of prion in a rod-like form; the prion rods being also termed scrapie-associated fibrils (SAF). Over the core is a layer of DNA, removable by DNAse; above the core is an outer protein coat which is digestible by a protease.

It would be desirable to have a method of diagnosis based on nucleic acid identification or on the core structure of the nemavirus protease-resistant protein in a living human or animal. Such methods have been suggested where a probe of DNA derived from the gene sequence coding for a prion protein are used. However, since it is well known that prion protein is encoded by a normal chromosomal gene found in all mammals, including those affected by encephalopathies, the above work has not gained acceptance. PCT Patent Application WO89/11545 (Institute for Animal Health Ltd) purports to describe a method of detection of scrapie susceptibility by use of a restriction fragment length polymorphism (RFLP) linked to the so called Sinc gene associated with short incubation times of sheep infected by scrapie. The RFLP is said to be located in a non-coding portion associated with the gene for the prion. At best, this method would detect only sheep with the short incubation time characteristic. Hitherto, methods of diagnosis based on nucleic acid identification have not been very successful or are likely to be unsuccessful, since an encephalopathy specific nucleic acid has eluded detection despite numerous attempts.

In human CJD cases, infectivity associated with the neuro-degenerative disorder has been consistently shown by titration studies to be present in blood. Although the protein associated with the neuro-degenerative disorder is present in urine of CJD cases, there is no known technique of diagnosis based on urine.

UK patent 2258867, describes a method for the diagnosis of encephalopathy using animal tissue. This method includes the use of a scrapie-specific nucleic acid, part of which can be labelled and used as an oligonucleotide probe in a hybridisation assay. Alternately, a sequence from the scrapie-specific nucleic acid is used as a primer in a polymerase chain reaction to make sufficient quantities to allow detection by a restriction fragment length method.

### OBJECTS OF THE INVENTION

It is an object of the present invention to provide a method for monitoring liquids for disease-associated materials, which can be used for detection of materials associated with diseases such as cancer, autoimmune and neuro-degenerative disorders.

It is a further object of the present invention to provide non-invasive means for the detection of various materials associated with cancer, and autoimmune and other disorders.

It is a further object of the present invention to provide means for the detection of materials associated with autoimmune and other disorders at an earlier stage than is possible using techniques currently available (particularly where the etiology is unknown or difficult to determine).

### SUMMARY OF THE INVENTION

According to a first aspect of the present invention, there is provided a method of monitoring a liquid for the presence of disease-modified or associated proteins, comprising the steps of:

(a) contacting a sample of the liquid with a solid, non-buoyant particulate material having free ionic valencies so as to concentrate the disease-modified or associated proteins in the sample; and

(b) monitoring the resulting disease-modified or associated proteins concentrated on the particulate material.

The concentration of the disease-modified or associated proteins takes place as a result of aggregation thereof on the surface of the particulate material.

It is a preferred feature of the present invention that the sample of liquid body fluid comprises a urine sample or a sample of another body fluid (such as serum or cerebral spinal fluid) comprising detectable levels of a disease-modified protein or detectable levels of viral matter.

According to the present invention, the disease-modified protein is a protein or a fragment thereof which is modified due to a disease in a host body and which protein or fragment thereof is excreted as the disease process begins. For example, it is known that amyloid β-protein is derived from amyloid β-precursor protein which is encoded by a normal host gene mapped to chromosome 21. In Alzheimer's disease, amyloid β-precursor protein slices into 3 segments as the disease progresses, one of the segments, typically the middle segment, being amyloid β-protein (a 4 KDa protein which forms plaques as seen in brain sections of Alzheimer's patients). The remaining two segments of the amyloid precursor protein have not been demonstrated in brain tissue of Alzheimer's patients. In patients testing positive for Alzheimer's disease, the presence of C-terminal segments of the amyloid β-precursor protein, or other segments, may be shown. In contrast, the urine of patients testing negative for Alzheimer's disease will not contain segments of the amyloid β-precursor protein. Such protein modifications have been found to occur in both infectious and non-infectious diseases, such as cancer.

According to the present invention, when the disorder is Alzheimer's disease, the disease-modified protein is typically amyloid β-protein. Furthermore, when the disorder is multiple sclerosis, the disease-modified protein is typically myelin. When the disorder is a bovine spongiform encephalopathy or Creutzfeldt Jakob disease, the disease-modified protein is typically protease-resistant protein.

According to the present invention, viruses such as cytomegalovirus, papillomavirus or the AIDS virus excreted in urine may be detectable.

According to the present invention, the protein may be associated with neuro-degenerative disorder, such as a nemavirus which may be concentrated from a sample of a body fluid, such as urine, taken from the animal.

According to a further preferred feature of the present invention, the disorder may be Alzheimer's disease, multiple sclerosis or a spongiform encephalopathy. Furthermore, since disease modified proteins have been demonstrated in cancer, for example in cancer of the cervix, the method according to the present invention may also be applicable to the detection and subsequent diagnosis of various forms of cancer. Similarly, various viruses associated with certain cancers, growths etc. have also been demonstrated in urine samples.

As indicated, the disease-modified or associated protein is concentrated from a body fluid, such as urine, using a solid non-buoyant particulate material, a preferred example of which is calcium phosphate. Calcium phosphate is widely used in transformation experiments to allow the introduction of DNA into a living cell, wherein it causes the precipitation of DNA. However, it has not been previously suggested for the purpose of concentrating a disease-modified or associated protein in a diagnostic sample of urine or the like.

A further example of a solid non-buoyant particulate material is hydroxyapatite or the like. Hydroxyapatite and similar media, as ion-exchange chromatography media, have previously been used to purify and concentrate viruses and their related soluble antigens. Such media have had limited application in the clinical diagnosis of human and animal diseases largely due to the impracticality of handling large sample volumes with slow transit of liquids through an ion-exchange column. A further problem is the low concentration of disease-related proteins which are in competition with contaminating proteins for exchange sites on the particulate materials. The method according to the present invention overcomes some of these difficulties by use of a medium which discriminates adsorption of albumin (in other words, proteins such as albumin are selectively not complexed as the medium is caused to lose the charge that allows albumin to complex).

The particulate material is preferably in the form of granules. Part of the disease-modified or associated protein, for example a protein associated with neuro-degenerative disorder (in the case of spongiform encephalopathies) or amyloid precursor protein APP (in the case of a non-transmissible neuro-degenerative disease, such as Alzheimer's and basic myelin protein oligocyte for multiple sclerosis), is thought to bind to the surface of the granules.

The steps leading to the concentration of the disease-modified or associated protein from a sample of body fluid such as urine typically comprise:

(a) collecting and centrifuging a sample of urine from an infected animal;

(b) collecting the supernatant produced following centrifugation of the sample of urine;

(c) adding a buffer and a solid, non-buoyant particulate material having free ionic valencies (such as calcium phosphate granules) to the supernatant;

(d) centrifuging the resulting mixture of buffer, particulate material and supernatant;

(e) collecting particulate material following centrifugation;

(f) adding a buffer to the particulate material;

(g) centrifuging the mixture of buffer and particulate material;

(h) collecting the particulate material;

(i) adding a buffer to the particulate material;

(j) centrifuging a mixture of the buffer and the particulate material; and

(k) collecting the particulate material containing the disease-modified or associated protein.

The sample of urine or the like can be concentrated 100 fold or more using calcium phosphate or other non-buoyant particulate material in the method according to the invention; the concentrated urine can then be used in several ways to allow diagnosis of diseases such as cancer, autoimmune and neuro-degenerative disorders.

According to a further aspect of the present invention, there is provided a method of monitoring a liquid for the presence of disease-modified or associated proteins, comprising the steps of:

(a) providing a sample of the liquid;

(b) passing the sample through a solid filter medium having free ionic valencies so as to complex at least one biological material to the medium, the biological material being selected from the group consisting of disease-modified or associated protein, a fragment thereof, a virus or a fragment thereof; and

(c) monitoring at least a part of the complexed biological material, wherein the presence of at least a part of the biological material is indicative of an association of the liquid with the relevant disease.

According to the present invention, the filter medium preferably comprises a sheet-like member with a pore size ranging from 1 to 100 microns. The pore size of the filter may be varied according to the size of the particles to be entrapped. Furthermore, the filter preferably comprises a gauze and/or cotton fiber.

In an alternate embodiment of the present invention, a non-buoyant particulate material having free ionic valencies(such as calcium phosphate) may be used in addition to the filter medium.

According to one aspect of the present invention, the concentrated or filtered sample of body fluid such as urine can be used for the detection of disease-modified or associated proteins using electron microscopy. In such a method, a grid is brought into contact with the sample of concentrated or filtered urine or the like and then the grid is fixed and stained. For example, the tubulofilamentous particles that are characteristic of the nemavirus associated with neuro-degenerative disorder may be visualized by electron microscopy.

Diagnosis can alternatively be carried out by means of, for example, an enzyme-linked immunosorbent assay (ELISA). The ELISA technique can be automated to provide a semi-quantitative result. The calcium phosphate for the concentration of the disease-modified or associated protein would be included as part of an ELISA kit. Such a kit according to the invention preferably further comprises a blocking buffer, an antibody to the disease-modified or associated protein and an antibody conjugate. A kit according to the invention preferably comprises:

(a) a solid, non-buoyant particulate material having free ionic valencies (such as calcium phosphate) in a form capable of complexing with protein present in a body fluid;

(b) a blocking buffer capable of complexing with any of the particulate material not complexed with the protein;

(c) a first antibody material capable of complexing with the complexed protein; and

(d) a further antibody which is capable of complexing with the first antibody.

When the neuro-degenerative disorder is a spongiform encephalopathy, an antibody to PrP may be added which will bind to the protein associated with the neuro-degenerative disorder on the surface of the particulate material. This is generally followed by a second antibody which will bind to the previous antibody, the second antibody being conjugated to a marker enzyme to allow detection of the protein associated with the neuro-degenerative disorder.

### BRIEF DESCRIPTION OF THE DRAWINGS

The use of calcium phosphate as an exemplary particulate material in the concentration of the disease-modified or associated protein and the subsequent detection using an ELISA method is shown schematically in FIGS. 1 to 6 of the accompanying drawings, which are by way of example only. In the drawings:

FIG. 1 shows a reaction vessel 1, having therein an exemplary calcium phosphate granule 2 and a disease-modified or associated protein 3;

FIG. 2 shows the disease-modified or associated protein 3 concentrated on the surface of the calcium phosphate granule 2;

FIG. 3 shows the unbonded sites on the surface of the calcium phosphate granule 2 blocked on the addition of blocking buffer (such as milk) 4;

FIG. 4 shows the addition of a first antibody against the disease-modified or associated protein 5;

FIG. 5 shows binding of the first antibody 5 to the disease-modified or associated protein 3 which is still bonded to the surface of the calcium phosphate granule 2;

FIG. 6 shows antibody detection using a second antibody 6 conjugated to a marker enzyme such as horseradish peroxidase or alkaline phosphatase; and

FIG. 7 is a photograph of a stained blot obtained in an exemplary diagnostic method according to the invention.

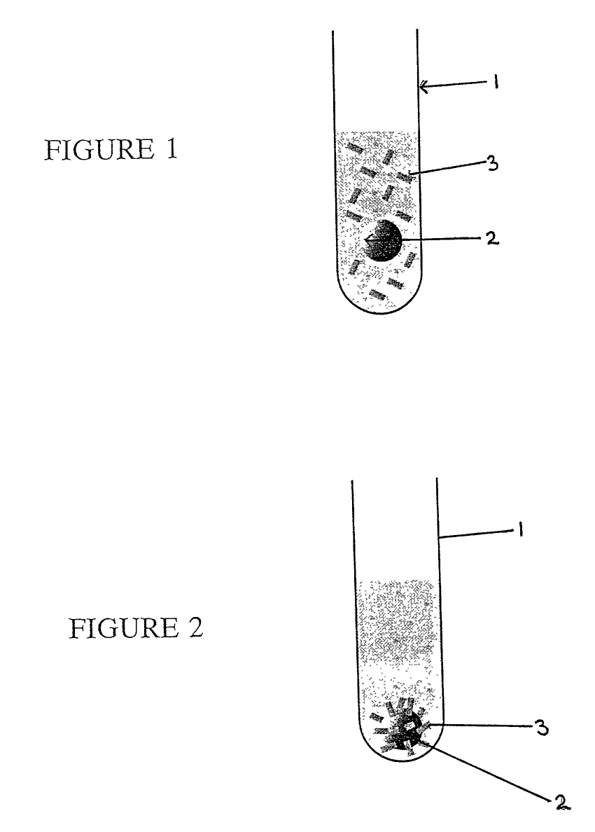

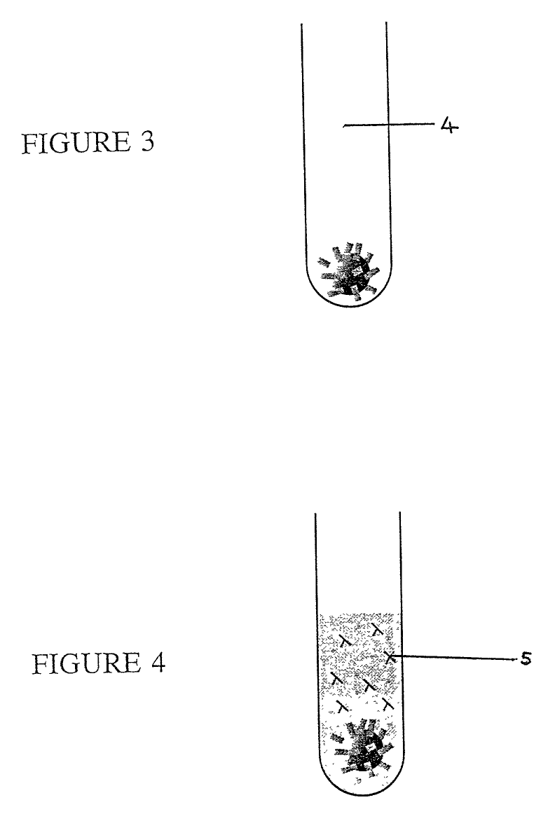

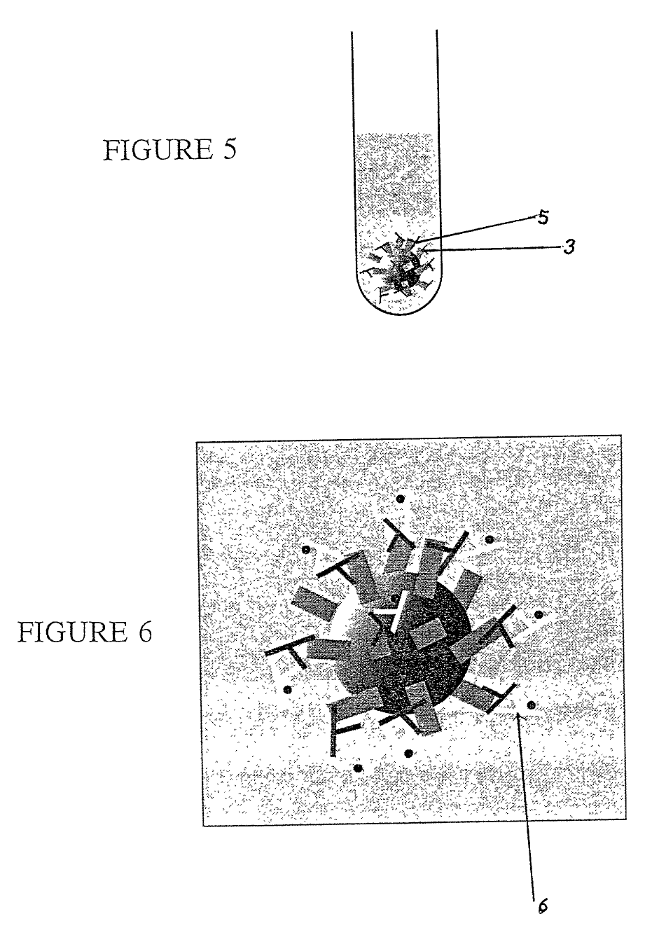

Another method for the diagnosis of diseases such as cancer, autoimmune and neuro-degenerative disorders from the concentrated or filtered sample of body fluid such as urine is to amplify the DNA in the sample by using a polymerase chain reaction (PCR).

In a preferred method for the diagnosis of encephalopathy, the palindromic oligonucleotide described above is used to amplify the sample DNA. Such oligonucleotides will not normally be longer than 200 nucleotides, even when used as probes; generally, they are likely to be very much shorter. Thus, for PCR purposes they are unlikely to comprise more than 24 nucleotides of the palindrome, plus an optional 5′-end or tail of (say) 8 to 20 nucleotides, making 32 to 44 nucleotides in all. The PCR will yield a product in the form of DNA of varying lengths containing the palindromic sequence. This can preferably be analyzed by a method relying on restriction by an enzyme.

The PCR product will produce bands of various molecular weights. In some instances the encephalopathy-specific DNA will be primed near its 3′-end, which will generate multiple copies of large molecules. The PCR product may be divided into two portions, of which the first may be run on a resolving gel to show a band of high molecular weight associated with the encephalopathy-specific DNA, the second portion being restricted with a restriction enzyme which cuts the palindromic sequence. This restriction will severely reduce the length of the longer DNA and eliminate certain other bands of shorter DNA altogether. Multiple restrictions of TACGTA will produce many bands of molecular weight too low to be detected. Restricted product can be compared with the unrestricted product, whereby disappearance of longer lengths of DNA upon restriction indicates the presence of the encephalopathy-specific DNA in the sample.

Examples of suitable restriction enzymes are SnaBI and AccI, which cut between the C and G of TACGTA and Bst11071 which cuts between A and T of one TACGTA sequence and the next TACGTA sequence. Such enzymes recognise the six-base sequence and leave blunt ends.

The sample of urine or other body fluid containing the concentrated disease-modified or associated protein can be used in a further assay for the diagnosis of diseases such as cancer, autoimmune and neuro-degenerative disorders, using a hybridization method. In the hybridization method, the sample of urine or the like, containing the disease-modified or associated protein, can be used as it is, or preferably, it may be amplified before use, for example, using a PCR method. The hybridization probe is preferably from 16 to 100 nucleotides long, especially about 40 nucleotides long. The hybridization assay can be carried out in a conventional manner; Southern blotting is preferred. For use in a hybridization assay, the oligonucleotide will normally be used in a labelled form, labelling being by any appropriate method such as radiolabelling, for example, by ³²P or ³⁵S, or by biotinylation (which can be followed by reaction with labelled avidin). However, it is also possible to use an unlabelled oligonucleotide as a probe provided that it is subsequently linked to a label. For example, the oligonucleotide could be provided with a poly-C tail which could be linked subsequently to labelled poly-G.

An alternative method for the diagnosis of diseases such as cancer, autoimmune and neuro-degenerative disorders is using a protein blotting method (Western blotting) which comprises detecting the protein of interest on the surface of a membrane (such as nitrocellulose) and detection of the protein using antibody technology.

The present invention has been described with particular reference to purification and detection of protein and viral matter from samples of body fluid such as urine. According to a further embodiment of the present invention, the solid non-buoyant particulate material may be used to concentrate viral samples form water, and/or the filter technology may be used to purify viral samples from water. The method according to the invention may prove useful in the detection of viral and/or bacterial matter from sea water, swimming pool water, tap water or the like.

### EXAMPLES

### Purification of a Disease-Modified or Associated Protein from a Sample (for Example Urine)

A sample of urine was collected from an animal suspected of having neuro-degenerative disorder. The urine sample was centrifuged and the supernatant collected. A suitable buffer and calcium phosphate granules were then added to the supernatant. This mixture of urine supernatant, buffer and calcium phosphate was allowed to rest at room temperature (with regular mixing) for at least ten minutes. The mixture was then centrifuged and the calcium phosphate granules collected. A suitable buffer was then added to the calcium phosphate granules followed by a further centrifugation step. The calcium phosphate granules were collected and the above addition of buffer and centrifugation step was repeated a further two times. The calcium phosphate granules were collected for the detection of a possible protein associated with a neuro-degenerative disorder using any of examples A, B, C, D, E or F detailed below.

### Example A

### Enzyme Linked Immunosorbent Assay

The calcium phosphate granules obtained following the above purification stage were used.

A suitable blocking buffer (for example milk) was added to the calcium phosphate granules and the solution was left mixing for at least sixty minutes. The solution was then centrifuged and the supernatant discarded. To the calcium phosphate granules that remain some phosphate buffered saline (PBS) containing Tween 20 was added and this was followed by a further centrifugation step. The above PBS-Tween 20 wash step was repeated at least four times. A first antibody was then added to the calcium phosphate granules. This was left to stand for at least 60 minutes with mixing at regular intervals. PBS-Tween 20 was added and followed by a centrifugation step. The supernatant was discarded and the PBS-Tween 20 wash step repeated at least four times. A second antibody, (one conjugated to a marker enzyme) was then added to the calcium phosphate granules and left mixing for at least sixty minutes. PBS-Tween 20 was then added followed by a centrifugation step. The supernatant was discarded and the wash step repeated with PBS-Tween 20 at least four times.

A substrate suitable for detection of the marker enzyme on the second antibody was then added. This was left to stand for at least twenty minutes and the reaction stopped by addition of a suitable reagent, such as concentrated sulfuric acid. Following centrifugation, the supernatant was collected and read photometrically at a suitable wavelength.

### Example B

### Preparation of Grids for Electron Microscopy

The calcium phosphate granules obtained following the purification stage were used.

Ethylenediaminetetraacetic acid (EDTA) was added to the calcium phosphate granules and mixed until a clear solution was produced. A carbon-coated grid was lowered into tubes containing some distilled water making sure the carbon/Formvar film was facing upwards. For each specimen at least two grids were prepared using the clear solution which was then transferred into the tube whilst gently rinsing off the distilled water. The grids were then centrifuged horizontally. After the centrifugation step sodium dodecyl sulfate was added and the grids transferred into distilled water. The grids were then washed several times in distilled water. The water was then momentarily replaced with glutaraldehyde containing ruthenium red. This solution was then rinsed out with distilled water and the grids were then momentarily introduced to a solution of osmic acid containing ruthenium red. The grids were again rinsed several times with distilled water. After the final wash of water with a drop of phosphotungstic acid the grids were dried and examined under an electron microscope.

### Example C

### Polymerase Chain Reaction

Again the calcium phosphate granules obtained following the purification stage were used.

EDTA was added to the calcium phosphate granules until a clear solution was produced. Some of this clear solution was taken and incubated with proteinase K for at least one hour at 55° C. The proteinase K was then heat inactivated at 95° C. and the solution used as a template in a polymerase chain reaction (PCR) A dNTP mix, primers, a buffer and AmpliTaq DNA polymerase were then added to the reaction mixture. Thirty cycles of PCR were carried out comprising a denaturation stage, annealing of primers and an extension stage. The PCR product was then cut with the restriction enzyme SnaB1. Cut and uncut PCR product was then analyzed using electrophoresis and the fragments visualized on the agarose gel after staining with ethidium bromide.

### Example D

### Protein Blotting for Immunoassay

Bio-Dot apparatus was used for the immunoblotting procedure. Nitrocellulose membranes were pre-wetted in Tris saline buffer (TSB) prior to placing in the bio-dot apparatus. After re-hydrating the membrane the wells of the apparatus were filled with antigen. The antigen solution being the clear solution produced on mixing the calcium phosphate granules (obtained from the purification stage) with EDTA. The entire antigen sample was allowed to filter through the membrane. After the antigen samples had completely drained from the apparatus the Tris saline buffer (TSB) was added and the liquid allowed to filter through. Blocking solution was then added to each well and the liquid allowed to filter through the apparatus. Tween-tris saline buffer (TTSB) wash solution was added to the apparatus and the flow valve adjusted to produce a vacuum to pull the wash solution through the membrane. The vacuum was then disconnected and a first antibody solution added to each sample well. The solution was allowed to filter through the membrane and the vacuum was re-applied to remove any excess liquid from the sample wells. TTSB wash solution was then added and pulled through the membrane with the aid of a vacuum. The wash process was then repeated three times.

Conjugated antibody solution was added to each well and the liquid allowed to filter through. TTSB wash solution was then added to each well and the solution pulled through the membrane with the aid of a vacuum. This wash process was repeated twice. The membrane was removed and placed in the colour development vessel. The membrane was then removed and washed with TSB to remove excess Tween 20. The membrane was then incubated in a suitable substrate until the development of spots were seen. After this time the membrane was rinsed in distilled water and photographed.

### Example E

### Southern Blotting

Again the calcium phosphate granules obtained following the purification stage were used.

Concentrated NaOH and DMSO was added to the calcium phosphate granules. The solution was mixed and heated and then cooled down to room temperature after which concentrated ammonium acetate was added. Nitrocellulose membrane was then wetted in 6XSSC and the bio-dot apparatus assembled. The DNA sample was applied and allowed to filter through the membrane. After the sample had filtered 2XSSC was added to each well and vacuum applied to remove the liquid. The blot membrane was removed and washed in 2XSSC. The nitrocellulose membrane was then baked under vacuum before hydridization.

### Example F

### Western Blotting

The calcium phosphate granules obtained following the purification steps outlined were used.

Sodium dodecyl sulfate containing proteinase k was then added to the calcium phosphate granules and the mixture incubated for at least 60 minutes at 55° C. β-mercaptoethanol was added after the incubation period and the mixture was then boiled. Following this polyacrylamide gel electrophoresis was carried out. Proteins on the polyacrylamide gel were then transferred to a nitrocellulose membrane. The membrane was air dried and then washed in tris buffered saline. Any unabsorbed sites were then blocked using heat inactivated horse serum and goat milk. A first antibody made up in tris-buffered saline containing Tween 20 and milk was then applied to the membrane which was left to incubate for at least one hour. The membrane was then washed several times. A second antibody conjugated to a marker enzyme (which was also made up in a solution of tris-buffered saline containing Tween 20) was then applied to the membrane. This was left to incubate for at least 60 minutes and then washed in a solution of tris buffered saline to remove excess Tween 20. The membrane was then incubated in a suitable substrate until the development of bands were seen. After this time the membrane was rinsed in distilled water and photographed.

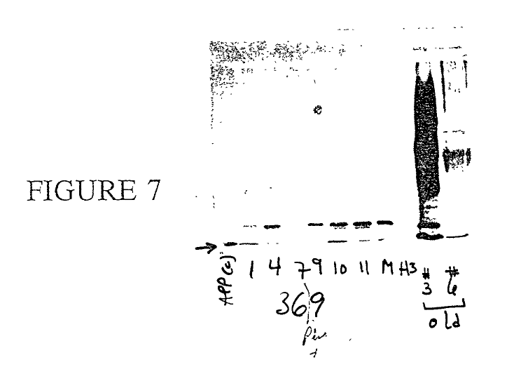

In an exemplary method, beta-amyloid protein (APP) was concentrated from urine specimens of patient having Alzheiemer's by the method described above and a Western blot performed. The resulting blot, stained by APP-antibody 369, is shown in FIG. 7 of the accompanying drawings. Positive results are seen in lane 0, control APP, lanes 1,3,4,6,9,10,11 and M from specimens from Alzheimer's patients.

Lane 3 is control and lane 7 relates to an assay for specimens from patients with Parkinson's disease.

### Detection of Amyloid Precursor Protein Segments in Alzheimer's Patients

One hundred ml, or larger, urine specimens, were collected in 50 ml tubes, three times, from 10 clinically diagnosed Alzheimer's patients and 10 healthy individuals of similar age group and sent fresh to the laboratory. After centrifugation at 1000 g for 10 minutes to remove gross debris, the supernatant was transferred to fresh 50 ml polypropylene centrifuge tubes. One 50 ml aliquot of the specimens was used and the rest frozen. To each tube, 1 ml buffer was added, mixed and then 500 μl non-buoyant particulate flock added. Tubes were left on a roller for 30 minutes at room temperature and agitated every 10 minutes. The tubes were then centrifuged at 200 g for 3 minutes and the pellet collected and supernatant discarded. The pellet of non-buoyant particulate flock with protein fragments adsorbed was transferred to a microfuge tube and suspended with another 1 ml buffer and centrifuged. This step was repeated twice. Following concentration of the urine, buffer was removed by centrifugation at 10,000 g for 1 minute and 250 μl sample buffer (3×) was added, mixed and followed by boiling for 3 minutes. The supernatant was collected into a fresh tube after centrifugation at 10,000 g for 1 minute. This sequence provides an approximate concentration of 200 times.

### Western Blotting

After boiling, the samples were run on sodium dodecyl sulfate polyacrylamide gel electrophoresis (SDS-PAGE) gels. For each run, 20 μl of the 250 μl of the concentrate was loaded. Electrophoresis was carried out using 10% polyacrylamide gel using BIO-Rad mini-gel apparatus. Secretory amyloid precursor protein C-terminal was used for the control. After the run, the proteins were transferred to PVDF membrane. Unadsorbed sites were then blocked using milk blocking buffer with sod-azide. A first amyloid precursor protein antibody 369 was made up in blocking buffer which was left to incubate for one and a half hours. The membrane was then washed three times in wash buffer. A second antibody, conjugated to a marker enzyme, (which was also made up in secondary blocking buffer without sod-azide) was left to incubate for one and a half hours and then washed three times in wash buffer without sod-azide. Developing: 1 part of A+1 part of B on membrane for 1 minute. The liquid was blotted and the membrane exposed for 30 seconds and 5 minutes and the film developed.

### Results

Western immunoblots prepared from urine concentrates of all Alzheimer's patients showed positive reactivity to the antibody raised to the amyloid precursor protein segments. Samples include collection and processing on different days from the same patients. Apart from quantitative differences, in most cases, two bands of 27 to 30 KD and 7 KD were seen. In some patients, there was a third band, just below the 27 to 30 KDa band. None of these bands were seen in one patient with Parkinson's disease also included in this study. No bands were seen in control cases. For comparative purposes, urine specimens from some Alzheimer's disease cases were run in SDS-PAGE gel without concentration. None of the bands were seen in SDS-PAGE gel in these runs.

### Purification of Viral Samples from Water

Water samples were collected from laboratory tap and also from the River Tyne in gallon containers. A 2 to 5% suspension of faeces which contained rotavirus was prepared in PBS. One ml of the suspension was added into one gallon water sample, mixed by shaking for 2-3 minutes. To each container, 10 ml buffer was added, mixed and then the cap of the container was replaced with a ion-exchange filter. The liquid was poured by gently tilting the container and was discarded. The filter paper was removed and immersed in 250 μl saturated versene. Following the concentration 50 μl of versene was used to prepare the grids by low speed centrifugation technique (Narang et al, 1980, Lancet, i, 1192-1193). The grids were stained with PTA and examined with an electron microscope. Rotavirus was found in all water samples with added faecal suspension, both in the tap and river concentrated by filter method. The filter method can be used to concentrate virus from river, sea and swimming pools water. The number of virus particles seen by an electron microscope demonstrated that the concentrated water samples could be used for analysis by Western Blotting.

What is claimed is:

1. A method of monitoring a liquid for the presence of disease-modified or associated proteins, comprising the steps of:
(a) contacting a sample of said liquid with a solid, non-buoyant particulate material having free ionic valencies so as to concentrate said disease-modified or associated proteins in said sample; and
(b) monitoring the resulting disease-modified or associated proteins concentrated on said particulate material.

2. A method according to claim 1, wherein said liquid is a sample of body fluid taken from an animal.

3. A method according to claim 2, wherein said sample of body fluid is urine.

4. A method according to claim 1, wherein said particulate material comprises calcium phosphate in granular form.

5. A method according to claim 1, wherein said concentrated proteins are monitored using electron microscopy.

6. A method according to claim 1, wherein said concentrated proteins are monitored using an enzyme linked immunosorbent assay (ELISA).

7. A method according to claim 6, in which a first antibody is added to said concentrated proteins so as to permit said first antibody to complex with said concentrated proteins.

8. A method according to claim 7, wherein a second antibody which is conjugated to a marker enzyme is added to said complexed proteins so as to permit said second antibody to complex to said first antibody.

9. A method according to claim 1, wherein said concentrated proteins are amplified using a polymerase chain reaction and then monitored by a restriction fragment length method.

10. A method according to claim 1, wherein said concentrated proteins are used in a hybridization reaction and then monitored using Western blotting.

11. A kit for carrying out an ELISA reaction, the kit comprising:
(a) a solid, non-buoyant particulate material having free ionic valencies in a form capable of complexing with disease-modified or associated proteins present in a sample of liquid;
(b) a blocking buffer capable of complexing with said particulate material not complexed with said proteins;
(c) a first antibody material capable of complexing with said complexed proteins; and
(d) a further antibody which is capable of complexing with said first antibody.

12. A kit according to claim 11, wherein said liquid is a sample of body fluid taken from an animal.

13. A kit according to claim 12, wherein said sample of body fluid is urine.

14. A kit according to claim 11, wherein said particulate material comprises calcium phosphate in granular form.

15. A method for concentrating disease-modified or associated proteins from a sample of liquid which comprises the following steps:
(a) collecting and centrifuging said sample of liquid;
(b) collecting the supernatant produced following centrifugation of said sample;
(c) adding a buffer and a solid, non-buoyant particulate material having free ionic valencies to said supernatant;
(d) centrifuging the resulting mixture of said buffer, said particulate material and said supernatant;
(e) collecting said particulate material following centrifugation;
(f) adding a buffer to said particulate material;
(g) centrifuging said mixture of said buffer and said particulate material;
(h) collecting said particulate material;
(i) adding a buffer to said particulate material;
(j) centrifuging a mixture of said buffer and said particulate material; and
(k) collecting supernatant containing the disease-modified or associated proteins.

16. A method according to claim 15, wherein said liquid is a sample of body fluid taken from an animal.

17. A method according to claim 16, wherein said sample of body fluid is urine.

18. A method according to claim 15, wherein said particulate material comprises calcium phosphate in granular form.

19. A method of monitoring a liquid for the presence of biological material selected from the group consisting of disease-modified or associated proteins, a fragment thereof, a virus or a fragment thereof, comprising the steps of:
(a) providing a sample of said liquid;
(b) passing said sample through a solid filter medium having free ionic valencies so as to complex at least one of said biological material to said medium; and
(c) monitoring at least a part of said complexed biological material, wherein the presence of at least a part of said biological material is indicative of an association of said liquid with the relevant disease.

20. A method according to claim 19, wherein said liquid is a sample of body fluid taken from an animal.

21. A method according to claim 20, wherein said sample of body fluid is urine.

22. A method according to claim 19, wherein said filter comprises a gauze fiber material.

23. A method according to claim 19, wherein said filter comprises a cotton fiber material.

24. A method according to claim 19, wherein said filter medium comprises a sheet-like member with a pore size ranging from 1 to 100 microns.

25. A method according to claim 19, wherein said complexed biological material is monitored using electron microscopy.

26. A method according to claim 19, wherein said complexed biological material is monitored using an enzyme linked immunosorbent assay (ELISA).

27. A method according to claim 26, in which a first antibody is added to said complexed biological material so as to permit said first antibody to complex with said complexed biological material.

28. A method according to claim 27, wherein a second antibody which is conjugated to a marker enzyme is added to said complexed biological material so as to permit said second antibody to complex to said first antibody.

29. A method according to claim 19, wherein said complexed biological material is amplified using a polymerase chain reaction and then monitored by a restriction fragment length method.

30. A method according to claim 19, wherein said complexed biological material is used in a hybridization reaction and then monitored using Western blotting.

31. A method of monitoring a liquid for the presence of biological material selected from the group consisting of disease-modified or associated proteins, a fragment thereof, a virus or a fragment thereof, comprising the steps of:
(a) providing a sample of said liquid;
(b) contacting said sample with a solid, non-buoyant particulate material having free ionic valencies;
(c) centrifuging at least once, said mixture of said particulate material and said sample;
(d) collecting the supernatant and passing said supernatant through a solid filter medium having free ionic valencies so as to complex at least one of said biological material to said medium; and
(e) monitoring at least a part of said complexed biological material, wherein the presence of at least a part of said biological material is indicative of an association of said liquid with the relevant disease.

32. A method according to claim 31, wherein said liquid is a sample of body fluid taken from an animal.

33. A method according to claim 32, wherein said sample of body fluid is urine.

34. A method according to claim 31, wherein said particulate material comprises calcium phosphate in granular form.

35. A method according to claim 31, wherein said filter comprises a gauze fiber material.

36. A method according to claim 31, wherein said filter comprises a cotton fiber material.

37. A method according to claim 31, wherein said filter medium comprises a sheet-like member with a pore size ranging from 1 to 100 microns.

38. A method according to claim 31, wherein said complexed biological material is monitored using electron microscopy.

39. A method according to claim 31, wherein said complexed biological material is monitored using an enzyme linked immunosorbent assay (ELISA).

40. A method according to claim 39, in which a first antibody is added to said complexed biological material so as to permit said first antibody to complex with said complexed biological material.

41. A method according to claim 40, wherein a second antibody which is conjugated to a marker enzyme is added to said complexed biological material so as to permit said second antibody to complex to said first antibody.

42. A method according to claim 31, wherein said complexed biological material is amplified using a polymerase chain reaction and then monitored by a restriction fragment length method.

43. A method according to claim 31, wherein said completed biological material is used in a hybridization reaction and then monitored using Western blotting.
